SEQUENCE LISTING (21 sequences)

SEQ ID NO: 1 - DNA, 28 bp, Artificial Sequence
  Synthetic
taatgctagcggcaaccacacgtgggag

SEQ ID NO: 2 - DNA, 19 bp, Artificial Sequence
  Synthetic
tccagttgtcgtggtgcag

SEQ ID NO: 3 - DNA, 21 bp, Artificial Sequence
  Synthetic
gttgaatattgcgatgccgaa

SEQ ID NO: 4 - DNA, 22 bp, Artificial Sequence
  Synthetic
tccttgttttccgtggctttat

SEQ ID NO: 5 - DNA, 21 bp, Artificial Sequence
  Synthetic
ttcggctacctgcagatcaag

SEQ ID NO: 6 - DNA, 19 bp, Artificial Sequence
  Synthetic
ggtagcggtccacggagat

SEQ ID NO: 7 - DNA, 18 bp, Artificial Sequence
  Synthetic
gaaggtgaaggtcggagt

SEQ ID NO: 8 - DNA, 20 bp, Artificial Sequence
  Synthetic
gaagatggtgatgggatttc

SEQ ID NO: 9 - DNA, 28 bp, Artificial Sequence
  Synthetic
taatgctagcggcaaccacacgtgggag

SEQ ID NO: 10 - DNA, 19 bp, Artificial Sequence
  Synthetic
tccagttgtcgtggtgcag

SEQ ID NO: 11 - DNA, 18 bp, Artificial Sequence
  Synthetic
gaaggtgaaggtcggagt

SEQ ID NO: 12 - DNA, 20 bp, Artificial Sequence
  Synthetic
gaagatggtgatgggatttc

SEQ ID NO: 13 - DNA, 19 bp, Artificial Sequence
  Synthetic
ctacctggctgtggctcat

SEQ ID NO: 14 - DNA, 21 bp, Artificial Sequence
  Synthetic
caaagacgcggtatagattca

SEQ ID NO: 15 - DNA, 21 bp, Artificial Sequence
  Synthetic
tgatgggtgtgaaccaagaca

SEQ ID NO: 16 - DNA, 19 bp, Artificial Sequence
  Synthetic
ccagtggatcagggatgat

SEQ ID NO: 17 - DNA, 30 bp, Artificial Sequence
  Synthetic
caggaattctcggcaaccacacgtgggagg

SEQ ID NO: 18 - DNA, 29 bp, Artificial Sequence
  Synthetic
cgctctagagccactcggggttcattgtg

SEQ ID NO: 19 - DNA, 25 bp, Artificial Sequence
  Synthetic
cagatgcataaacgctcaactttgg

SEQ ID NO: 20 - DNA, 27 bp, Artificial Sequence
  Synthetic
gtcggtacctgtgctggcggcagcatc

SEQ ID NO: 21 - protein, 8 aa, Artificial Sequence
  Synthetic peptide
CHTQADSC

(19) United States
(12) Patent Application Publication
(10) Pub. No.: US 2002/0107197 A1
(43) Pub. Date: Aug. 8, 2002

(54) Ligands for G protein coupled receptors and methods of using them

(76) Inventors: Yan Xu, Pepper Pike, OH (US); Kui Zhu, Evanston, IL (US)

Correspondence Address: Janet E. Reed, Esq., WOODCOCK WASHBURN LLP, One Liberty Place - 46th Floor, Philadelphia, PA, 19103, US

(21) Appl. No.: 09/956,636

(22) Filed: Sep. 20, 2001

Related U.S. Application Data
(60) Provisional application No. 60/234,249, filed on Sep. 20, 2000.

(51) Int. Cl. A61K 38/16

(52) U.S. Cl. 514/12

(57) ABSTRACT

The present invention is directed to assays for potential drugs which modulate SPC and/or LPC binding to GPCRs, diagnostic assays for disease conditions associated with expression of the receptors and ligands, methods of causing cellular effects by modulating such binding, methods of treating disease conditions associated with SPC and/or LPC expression or GPCR expression, and synthetic peptide analogs which bind to GPCRs.

### CROSS REFERENCE TO RELATED APPLICATIONS

This application claims the benefit of co-owned earlier filed U.S. patent application Ser. No. 60/234,249, filed Sep. 20, 2000, which application is incorporated by reference as if fully set forth herein.

### TECHNICAL FIELD OF THE INVENTION

The present invention relates generally to the fields of genetics and cellular and molecular biology. More particularly, the invention relates to ligands to particular G protein coupled receptors, and methods based on the interactions of the receptors and ligands.

### BACKGROUND OF THE INVENTION

The G protein-coupled receptors (GPCRs) form a vast superfamily of cell surface receptors which are characterized by an amino-terminal extracellular domain, a carboxyl-terminal intracellular domain, and a serpentine structure that passes through the cell membrane seven times. Hence, such receptors are sometimes also referred to as seven transmembrane (7 TM) receptors. These seven transmembrane domains define three extracellular loops and three intracellular loops, in addition to the amino- and carboxy-terminal domains. The extracellular and transmembrane portions of the receptor have a role in recognizing and binding one or more extracellular binding partners (e.g., ligands), whereas the intracellular portions have a role in recognizing and communicating with downstream molecules in the signal transduction cascade.

The G protein-coupled receptors bind a variety of ligands including calcium ions, hormones, chemokines, neuropeptides, neurotransmitters, nucleotides, lipids, odorants, and even photons, and are important in the normal (and sometimes the aberrant) function of many cell types. (See generally Strosberg, Eur. J. Biochem. 196:1-10 (1991) and Bohm et al., Biochem J. 322:1-18 (1997)). When a specific ligand binds to its corresponding receptor, the ligand typically stimulates the receptor to activate a specific heterotrimeric guanine-nucleotide-binding regulatory protein (G-protein) that is coupled to the intracellular portion of the receptor. The G protein in turn transmits a signal to an effector molecule within the cell, by either stimulating or inhibiting the activity of that effector molecule. These effector molecules include adenylate cyclase, phospholipases and ion channels. Adenylate cyclase and phospholipases are enzymes that are involved in the production of the second messenger molecules cAMP, inositol triphosphate and diacyglycerol. It is through this sequence of events that an extracellular ligand stimuli exerts intracellular changes through a G protein-coupled receptor. Each such receptor has its own characteristic primary structure, expression pattern, ligand-binding profile, and intracellular effector system.

Because of the vital role of G protein-coupled receptors in the communication between cells and their environment, such receptors are attractive targets for therapeutic intervention, for example by activating or antagonizing such receptors. For receptors having a known ligand, the identification of agonists or antagonists may be sought specifically to enhance or inhibit the action of the ligand. Some G protein-coupled receptors have roles in disease pathogenesis (e.g., certain chemokine receptors that act as HIV co-receptors may have a role in AIDS pathogenesis), and are attractive targets for therapeutic intervention even in the absence of knowledge of the natural ligand of the receptor. Other receptors are attractive targets for therapeutic intervention by virtue of their expression pattern in tissues or cell types that are themselves attractive targets for therapeutic intervention. Examples of this latter category of receptors include receptors expressed in immune cells, which can be targeted to either inhibit autoimmune responses or to enhance immune responses to fight pathogens or cancer. This category of receptor is also useful as a marker for identifying and/or purifying (e.g., via fluorescence-activated cell sorting) cellular subtypes that express the receptor.

Thus, there is a need for identifying ligands to GPCRs, and for methods based on the interactions between the receptors and their identified ligands.

### SUMMARY OF THE INVENTION

Sphingosylphosphorylcholine (SPC) and lysophosphatidylcholine (LPC) are bioactive lipid molecules involved in numerous biological processes. The present inventors have discovered that SPC and LPC are natural ligands for at least four GPCRs, G2A, OGR1, GPR4 and TDAG8.

The present invention is directed to diagnostic, prognostic, and therapeutic methods for disease conditions associated with SPC and/or LPC expression or GPCR expression, and compositions, such as synthetic peptides, which bind to SPC and thereby interfere with the activation of its receptors. The invention also is directed to screening of compounds for use as drugs to modulate SPC and/or LPC binding to GPCRs.

In one aspect, then, the invention is directed to a method of suppressing tumor cell growth comprising contacting the tumor cell with an antagonist of GPR4 or TDAG8. The antagonist may be a synthetic peptide which binds to SPC, such as the peptide whose sequence is provided in SEQ ID NO. 21. The method may be performed in vivo in a human.

In another aspect, the invention provides a method of treating a disease condition in a patient comprising administering to the patient a therapeutically effective amount of an antagonist GPR4 or TDAG8. The antagonist may be a synthetic peptide which binds to SPC such as the peptide whose sequence is provided in SEQ ID NO. 21. In a particular aspect, the disease condition is Niemann-Pick disease type A or atopic dermatitis.

In another aspect, the invention provides a method of treating a disease condition in a patient comprising administering to the patient a therapeutically effective amount of an agent which interferes with GPR4 or TDAG8 binding to LPC. In a particular aspect, the disease condition is atherosclerosis, arthritis, liver cirrhosis, endometriosis, cancer, or Alzheimer's disease. The interfering agent may be lyso-PAF.

In another aspect, the invention provides a method of preventing a disease condition comprising administering to the patient a therapeutically effective amount of an agent which interferes with GPR4 or TDAG8 binding to LPC. In a particular aspect, the disease condition is an inflammatory disease condition selected from the group consisting of atherosclerosis, arthritis, liver cirrhosis, endometriosis, cancer, or Alzheimer's disease. The interfering agent may be lyso-PAF.

In another aspect, the invention provides a method of detecting the presence of a disease condition in a patient comprising measuring the level of SPC in the patient. In a particular aspect, the disease is ovarian cancer.

In another aspect, the invention provides a method of determining the progress of a disease condition in a patient comprising measuring the level of SPC in the patient. In a particular aspect, the disease is ovarian cancer.

In another aspect, the invention provides a method of determining whether a disease condition in a patient is benign comprising measuring the level of SPC in the patient. In a particular aspect, the disease is ovarian cancer.

In another aspect, the invention provides a method of modulating the activity of GPR4 comprising contacting the GPR4 with SPC or LPC.

In another aspect, the invention provides a method of modulating the activity of TDAG8 comprising contacting the TDAG8 with SPC or LPC.

In another aspect, the invention provides a method of screening a drug candidate comprising contacting the drug candidate with GPR4 or TDAG8 in the presence of SPC or LPC.

In another aspect, the invention provides a composition comprising a synthetic peptide capable of binding to SPC. The composition may be capable of interfering with the binding of SPC to a GPCR. The GPCR may be selected from OGR1, G2A, GPR4, and TDAG8. The composition may also comprise a pharmaceutically acceptable excipient.

### BRIEF DESCRIPTION OF THE FIGURES

FIG. 1 shows upregulation of LPC receptors in a time- and dose-dependent manner by LPC in Jurkat cells. Jurkat cells were treated with 0.1 and 1 μM LPC for various times (1, 2, 6, and 24 hours). At each time point, cells were collected and total RNA was extracted. Real-time quantitative PCR was utilized to determine relative amounts of GPR4 (shown in FIG. 1A and TDAG8 (shown in FIG. 1B) expressed under each condition. All PCR reactions were performed in triplicate.

FIG. 2 shows SPC levels were elevated in plasma from patients with ovarian cancer. Blood samples were collected in EDTA-containing tubes and plasma was obtained after centrifugation (1750 g, 10 min at room temperature). Lipid extraction and analysis are described in detail in Xiao et al., Ann N Y Acad Sci. 905: 242-59., 2000; Xiao et al., Anal Biochem. 290: 302-313, 2001).

FIG. 3 shows the levels of SPC and LPC in 15 pairs of ascites samples.

FIG. 3A shows the SPC and S1P levels in 15 pairs of ascites samples.

FIG. 3B shows the LPC levels in 15 pairs of ascites samples. Patients=ovarian cancer patients; controls=non-malignant diseases.

FIG. 4 shows phage 414 blocked the growth inhibitory effect of SPC on DNA synthesis in HEY ovarian cancer cells. SPC (10 μM) inhibited DNA synthesis in HEY ovarian cancer cells (control bars). Different phage preparations (#413 to 416) did not affect the DNA synthesis in HEY cells (the black bars).

### DETAILED DESCRIPTION OF THE INVENTION

SPC is a bioactive lipid molecule involved in many biological processes, where it acts as a signaling molecule. SPC regulates diverse cellular functions including both cell proliferation and growth inhibition, smooth muscle contraction, and wound healing. SPC has been shown to be abnormally elevated in certain pathological conditions, such as Niemann-Pick disease type A or atopic dermatitis. A specific, high-affinity receptor for SPC has been implicated in a number of studies, but was not identified (Meyer zu Heringdrof et al., Naunyn Schmiedebergs Arch Pharmacol. 354: 397-403, 1996; Van Koppen et al., Mol Pharmacol. 49: 956-61, 1996). The inventors previously identified OGR1 as a high-affinity receptor for SPC and OGR1 may play a role in tumor suppression.

LPC is also an important bioactive lipid. A significant elevation of LPC levels in cells and tissues under different diseases has been reported. LPC plays an important role in atherosclerosis and many inflammatory diseases. It may affect various aspects of a variety of cell types involved in atherosclerosis, including endothelial cells, smooth muscle cells, monocytes, macrophages and T cells. In particular, LPC functions as a chemoattractant for macrophage and T cells. The attachment of monocytes and T-lymphocytes to injured endothelium followed by their migration into the intima is one of the first and most crucial steps in lesion development, which may trigger cell-mediated immunity to atherogenesis. LPC also increases the production of T cell- and macrophage-derived cytokines, such as interferon-gamma (IFN-g), HB-EGF, and IL-1, which promote the progression of the lesion. However, the mechanisms of LPC function has not been studied intensively and receptors for LPC were not previously identified.

The inventors have identified G2A as the first high affinity receptor for LPC (Kabarowski et al., Science 293, 702-705 2001). The inventors have also identified OGR1 as the first specific high affinity receptor for SPC (Xu et al. (2000) Nat Cell Biol 2, 261-267, the entirety of which is incorporated by reference herein)

OGR1 shares homology with several GPCRs, including GPR4, G2A, T cell death associated GPCR8 (TDAG8), and the platelet activating factor (PAF) receptor (Heiber et al. (1995) DNA & Cell Biology 14, 25-35; Mahadevan et al. (1995) Genomics 30, 84-88; Choi et al. (1996) Cell Immunol 168, 78-84; Kyaw et al. (1998) DNA Cell Biol. 17, 493-500; Weng et al. (1998) Proc Natl Acad Sci USA 95, 12334-12339; Xu et al. (1996) Genomics 35, 397-402; Kyaw et al. (1998) DNA Cell Biol. 17, 493-500; Choi et al. (1996) Cell Immunol. 168,:78-84).

The present inventors have now discovered that GPR4 and TDAG8 are also receptors for SPC and LPC. These GPCRs represent novel targets for SPC- and LPC-related diseases. Particularly, GPR4 appears to mediate the growth stimulatory effect of SPC, and therefore, it may represent a target to block tumor growth and/or a target for the treatment of SPC-related diseases, such as Niemann-Pick disease type A or atopic dermatitis. The inventors have also now identified GPR4 as a receptor for LPC.

Further in accordance with the present invention, the inventors have found that TDAG8 is a receptor for SPC and LPC. The inventors have discovered that LPC and SPC stimulate cell migration in a GPR4- and TDAG8-dependent manner. The cell-migration induced by LPC is one of the most important physiological/pathological roles of LPC in atherosclerosis. These discoveries enable elucidation of the physiological and pathological role and mechanism of LPC. More importantly, these receptors represent novel targets for LPC-related diseases.

Both SPC and LPC induce increases in intracellular calcium concentration in GPR4-, but not vector-transfected, cells. These effects are insensitive to treatment with specific platelet activating factor (PAF) receptor antagonists, which indicates that they are not mediated through an endogenous PAF receptor. SPC and LPC bind to GPR4 in GPR4-transfected CHO cells with K_{d/SPC}=36 nM, and K_{d/LPC}=159 nM, respectively. Competitive binding is elicited only by SPC and LPC. Both SPC and LPC activate GPR4-dependent activation of serum response element (SRE) reporter and receptor internalization. Cells expressing GPR4 respond to both SPC and LPC, but not sphingosine-1-phosphate (S1P), PAF, psychosine (Psy), glucosyl-β1′1-sphingosine (Glu-Sph), galactosyl-β1′1-ceramide (Gal-Cer), or lactosyl-β1′1-ceramide (Lac-Cer) to activate extracellular signal-regulated kinase (ERK) MAP kinase in a concentration- and time-dependent manner. SPC and LPC stimulate DNA synthesis in GPR4-expressing cells. Both ERK activation and DNA synthesis stimulated by SPC and LPC are pertussis toxin (PTX)-sensitive, indicating the involvement of a Gᵢ-heterotrimeric G protein. In addition, GPR4 expression confers chemotactic responses to both SPC and LPC in Swiss 3T3 cells. Thus, GPR4 is a receptor with high affinity to SPC and low affinity to LPC, and multiple cellular functions can be transduced via this receptor. These receptors may belong to a subfamily and their ligands may be lysolipids containing the phosphorylcholine moiety shared by SPC and PAF (Xu et al. (2000) Nat Cell Biol 2, 261-267).

TDAG8-transfected cells respond to LPC and SPC to activate MAP kinase pathways. Both LPC and SPC activate the SRE-reporter system in TDAG8-transfected cells. In GPR4 and TDAG8-transfected cells, SPC and LPC bind to GPR4 and TDAG8. In addition, LPC causes internalization of the receptors in a structurally specific manner. Furthermore, the inventors have demonstrated that cells migrate in respond to LPC and SPC in a GPR4- and TDAG8-dependent manner.

The present inventors have found that LPC upregulates GPR4 and TDAG8 in Jurkat cells. Also, SPC levels were discovered to be elevated in ascites in patients with ovarian cancer, as compared to other benign diseases. The inventors have also identified a synthetic peptide which binds to SPC.

OGR1 and GPR4 may play both overlapping and distinct physiological and pathological roles. We have shown that OGR1 and GPR4 bind SPC with similar affinities (33 nM and 36 nM, respectively) and both receptors mediate SPC-induced increases in intracellular calcium and ERK activation. However, GPR4- and OGR1-mediated ERK activation is PTX-sensitive and -insensitive, respectively, suggesting that GPR4 and OGR1 couple to different G proteins to activate ERK. More importantly, these differential couplings appear to lead to differential effects on cell proliferation. While OGR1 mediates PTX-insensitive growth inhibition by SPC in a number of cells tested, GPR4 mediates PTX-sensitive DNA synthesis by SPC in Swiss 3T3 cells. Together, these data indicate that the endogenous receptor for SPC is GPR4-like, rather than OGR1-like, since parental cells respond to SPC to activate ERK and increase DNA synthesis through a PTX-sensitive pathway. The expression of GPR4 in these cells has been confirmed by quantitative PCR analysis.

GPR4 and OGR1 have different tissue distributions, which may be related to their physiological and pathological roles. Both OGR1 and GPR4 are highly expressed in the lung. However, OGR1 is expressed at high levels in the placenta, spleen, testis, small intestine and peripheral leukocytes, whereas GPR4 is not expressed, or is expressed at relatively low levels, in these tissues. GPR4 is expressed at high levels, however, in the liver, kidney, and ovary, while OGR1 is not expressed in these tissues.

GPR4 binds to LPC (and SPC), but not to PAF or lyso-PAF, to mediate an increase in intracellular calcium, receptor internalization, SRE activation, MAP kinase activation, DNA synthesis, and cell migration. Although effects of LPC on transmembrane signal transduction have been widely reported, a specific receptor recognizing LPC had not been identified previously. LPC lyses cells at high concentrations (>30 μM) and many of the cellular effects previously reported for LPC were observed at high concentrations. Therefore, it is possible that some of the LPC effects in vivo are not receptor mediated. However, evidence has been accumulating to support the notion that, at low concentrations, LPC acts through membrane receptors. First, at relatively low concentrations 10 μM), LPC exerts cell-specific effects. Second, LPC increases intracellular Ca²⁺ concentration in association with production of inositol phosphates. Third, these effects of LPC are markedly inhibited by treatment of the cells with PTX and U73122 (an inhibitor of phospholipase C). (Okajima et al. (1998) Biochemical Journal 336, 491-500). Some LPC effects are believed to be mediated through the PAF receptor in various cell types, reflected by their partial sensitivity to PAF receptor antagonists. We have found, however, that intracellular calcium increase and receptor internalization induced by LPC are dependent on the expression of GPR4 and are insensitive to the PAF receptor antagonists BN52021, WEB-2071 and WEB-2086. These results clearly demonstrate that LPC does not activate these signaling pathways through PAF receptors. Further, while the expression of G2A is restricted to lymphoid tissues, GPR4 is more ubiquitously expressed. This, together with the different affinities of these two receptors for LPC, may reflect distinct physiological functions for G2A and GPR4.

Physiological concentrations of LPC in body fluids, including blood and ascites, are relatively high (5-180 μM), when compared to other signaling lipid molecules, such as LPA, S1P and SPC. All receptors would be saturated, down regulated, and/or desensitized at these concentrations of LPC if it were all in a form available to its receptors. However, different concentrations of LPC present in various cellular and tissue systems (i.e. different LPC compartments) may regulate cellular functions differentially. LPC in plasma is present mainly in albumin- and lipoprotein-bound forms. These forms may be active in some non-receptor-mediated functions of LPC, such as delivery of fatty acids and choline, but may be in a form unavailable for receptor activation. It has been shown that some of the effects of LPC are decreased in the presence of albumin. Thus, the functionally available concentration of LPC in vivo, and the activation of LPC receptors may be controlled by the lower concentrations of free LPC. Our results support this notion. The presence of a 75-fold molar excess of BSA greatly diminished the ability of LPC to elicit an increase in [Ca²⁺]ᵢ through the GPR4 receptor. In vivo the theoretical molar ratio of albumin (approximately 3-5% in plasma) to LPC can be from 3- to 100-fold in plasma. In extravascular sites where albumin concentration is less than in plasma, the ratio of albumin to LPC can be lower.

TDAG8, which shares approximately 38% homology with OGR1 and GPR4, has recently been shown to be a Psy receptor (Im et al. (2001) J Cell Biol. 153, 429-3413). Treatment of cultured cells expressing this receptor with Psy or structurally related glycosphingolipids results in the formation of globoid, multinuclear cells. Observing the effect of Psy and related glycosphingolipids in calcium mobilization, competition of ligand binding, and MAP kinase activation assays produced no evidence that these lipids interact with GPR4.

Ligands of GPR4 induced cell shape changes, indicating that SPC and LPC may affect the cellular cytoskeleton. Both LPA and S1P are able to affect cytoskeleton through Rho. SPC and LPC are also able to activate Rho, as evidenced by C3-exoenzyme sensitivity of SRE reporter activity and cell migration induced by SPC/LPC.

SPC is a high-affinity, and LPC a lower-affinity, ligand for GPR4. This conclusion is directly derived from the results of ligand binding assays (K_{d} values of 36 vs. 159 nM for SPC and 16:0-LPC, respectively). This is also supported by results from assays of different signaling pathways activated by SPC and LPC, including increases in calcium, transcriptional activation of SRE, ERK activation, and stimulation of DNA synthesis and cell migration. In recent decades, many reports have described a significant elevation of LPC levels in cells and tissues in different diseases. Numerous lines of evidence suggest that LPC, which is a major lipid component of ox-LDL, and which accumulates in atherosclerotic lesions, plays pathological roles in the development of atherosclerosis and other chronic inflammatory diseases. LPC also plays other important biological roles. For example, LPC functions as a fatty acid and choline carrier and delivers fatty acids more specifically to brain than other tissues. The identification of GPR4 and TDAG8 as receptors for LPC and SPC solidifies the assignment of a new lysophospholipid receptor subfamily (OGR1, G2A, GPR4, and TDAG8).

Given the information set forth in the present disclosure, those of skill in the art will appreciate the many varied uses of the present invention. For example, in one embodiment of the present invention, GPR4 and TDAG8 are regulated through use of their identified ligands SPC and LPC to suppress tumor growth. The present inventors have discovered that the growth promoting activity of SPC is mediated through GPR4 and the growth inhibitory effect of SPC is mediated through OGR1. Tumor suppression may be achieved by regulating the levels and activities of these receptors. When GPR4 is highly expressed, it mediates SPC-stimulated DNA synthesis. For tumors expressing high levels of GPR4, neutralizing antibody, antisense, or ribozyme approaches can be applied to reduce the levels of GPR4. Such methods are well know in the art.

The detrimental growth-promoting features of the SPC-GPR4 interaction also may be regulated by eliminating or reducing the pathological concentrations of SPC in or near target cells comprising GPR4. In one embodiment, an antagonist against GPR4, such as a synthetic peptide which competes with SPC, may be used.

Amounts of the receptors in tumor samples may be analyzed using a quantitative PCR method developed by the present inventors, as described below in Example 1.

In another embodiment of the invention, the activity of these SPC and LPC receptors can be modulated by modulating the amount of their ligands, SPC and LPC. In addition, a number of other reagents are able to regulate GPR4 and TDAG8, including ceramide, phytohemagglutinin (PHA-P), N,N′,N′-tetrakis(2-Pyridylmethyl)-ethylenediamine (TPEN). These and other reagents, and in various combinations, can be used to down or up-regulate GPR4 and TDAG8.

Another embodiment of the present invention is a method of antagonizing TDAG8 activity. Coronary heart disease and atherosclerosis are the major types of cardiovascular diseases, which are the leading causes of death in developed countries. Inflammation plays a central role in the pathology of a variety of pathological conditions ranging from atherosclerosis, arthritis, liver cirrhosis, endometriosis, cancer and Alzheimer's disease. Under normal conditions the inflammatory response initiates protective actions, but triggers tissue damage under pathological conditions. LPC has been shown to be involved in the progression of atherosclerosis and many inflammatory diseases. In particular, numerous lines of evidence suggest that LPC plays important pathological roles in the development of atherogenesis. LPC is a major lipid component of oxidized low density lipoprotein (ox-LDL) and is formed through a phospholipase A₂ activity, an enzymatic activity inherent to apo B that prefers phosphatidylcholine as a substrate only after the oxidation of the unsaturated fatty acid in the sn-2 position. The present invention's discovery of LPC receptors will have important applications in treatment of atherosclerosis and many other inflammation-related diseases, such as arthritis, liver cirrhosis, endometriosis, cancer, and Alzheimer's disease.

The attachment of monocytes and T-lymphocytes to the injured endothelium followed by their migration into the intima is one of the first and most crucial steps in lesion development. TDAG8 mediates LPC- and SPC-induced cell migration. LPC is also able to upregulate TDAG8 and GPR4. Therefore, in another embodiment of the present invention, blocking LPC- and SPC-induced migration of T cells or macrophages is a novel way of intervention of the development of atherosclerosis. The inventors have found that PAF and lyso-PAF bind to and internalize TDAG8, but do not activate TDAG8. Therefore, these two lipids may function as antagonists of TDAG8. While PAF itself has numerous physiological effects, lyso-PAF is the inactive ligand for the PAF receptor. Therefore, lyso-PAF and/or a compound derived from lyso-PAF may be used physiologically in either prevention and/or treatment of atherosclerosis.

In yet another embodiment of the present invention, ovarian cancer may be detected, and differences between benign and malignant masses may be determined, by measuring phospholipid contents. The inventors have detected elevated levels of SPC in plasma and ascites from patients with ovarian cancer, when compared to samples from healthy controls or patients with non-malignant diseases. SPC may be used along with lysophosphatidic acid (LPA) and lysophosphatidylinositol (LPI) (previously identified markers for ovarian cancer), to detect early stage ovarian cancer.

In another embodiment of the present invention, SPC is used as a prognostic marker useful in monitoring disease progression. Another very important application of the markers is to differentiate benign from malignant masses. The ultrasound-based method is highly sensitive to detect abdominal mass. However, it is not specific enough to efficiently differentiate malignant and non-malignant masses, which resulted in a large number of unnecessary surgeries. The lipid tests, when used in combination with presently available cytology and CA125 tests, serves as an alternative to surgical exploration for those patients with a pelvic mass that are poor surgical candidates.

In another embodiment of the present invention, SPC levels are diminished by a synthetic SPC-binding peptide. SPC has been shown to be abnormally elevated in certain pathological conditions, such as Niemann-Pick disease type A or atopic dermatitis. Niemann-Pick disease is caused by disturbance of sphingolipid metabolism characterized by enlargement of liver and spleen, anemia, lymphadenopathy, and progressive mental and physical deterioration. It is a hereditary disease, with its onset in early infancy. SPC is also involved in inflammation. Since SPC is elevated in plasma and ascites form patients with ovarian cancer, one of skill in the art may reduce abnormal, pathological concentrations of SPC by administering a peptide that binds to SPC. Such peptides may be derived through screening a phage display library. The peptide disclosed herein as one embodiment of the present invention, derived from clone 414 (see Example 12), inverted the growth inhibitory effect of SPC through OGR1 in HEY cells. In another embodiment, the peptide may be found in a composition comprising other agents, such as a pharmaceutically acceptable excipient, many of which are known to those skilled in the art. This peptide and its derivatives may be chemically synthesized and tested in SPC binding assays, SPC clearance assays, and biological assays, such as SPC-induced growth inhibition. When verified through in vitro assays and in mouse models, this peptide, its derivatives, and others derived in a similar manner, may be used to remove pathological concentrations of SPC and to treat patients with Niemann-Pick disease type A or atopic dermatitis who have abnormal, high levels of SPC.

In another embodiment of the present invention, potential drugs may be screened for their effect on regulating the activity or expression of GPR4 and TDAG8, or the activity or expression of SPC and LPC, through assays based on the binding of the receptors to these ligands. For example, one may screen a candidate drug for inhibition of SPC or LPC binding to GPR4 or TDAG8, using, for example, radioligand binding assays as described below in Example 4 in the presence or absence of the candidate drug. Briefly, cells are transfected with vectors comprising the gene for GPR4 or TDAG8, and then radioactively labeled SPC or LPC are incubated with the transfected cells under conditions allowing the binding of SPC or LPC to the cells expressing the receptor. This is done in the presence or absence of the candidate drug compound. Cell-bound radioactively labeled SPC or LPC may then be collected and specific binding measured, thereby determining the effect on binding of the candidate drug compound. Alternative binding assays known in the art may also be used.

Other assays may be used to examine SPC and LPC mediated signal transduction, based on the known signal pathway involved in activating a GPCR (i.e., binding of G Protein), and thus such assays would be useful to investigate the effects of candidate drug compounds which act as modulators of signal transduction. Examples 5, 6, 7, and 8 below provide illustrative examples of cell-based assays involved in the signal transduction pathways for GPR4 and TDAG8, and would therefore be useful in testing candidate drug compounds for their effects thereon.

As shown in Example 10 below, LPC upregulates GPR4 and TDAG8 in Jurkat cells. Assays may therefore make use of this upregulation in testing putative drug compounds for their effects on such upregulation. That is, cells may be treated with LPC (or SPC), in the presence or absence of a candidate drug compound, and then assayed for expression of the GPR8 or TDAG8 receptors.

Candidate drug compounds may also be screened using an assay based on the fact that SPC levels are elevated in humans with ovarian cancer, as described below in Example 11. Briefly, a tumor-prone animal may be treated with the candidate drug compound, then induced to produce a tumor. Comparing to a control animal, one may measure the ease of inducing the tumor to determine the protective effect of the candidate drug compound, and where such a protective effect is seen, one may determine whether the animal's SPC level has decreased compared to the control animal. Alternatively, one may simply measure the SPC levels in the test and control animals to assay whether the candidate drug compound is likely to exert a protective effect.

The present invention is illustrated through the following nonlimiting Examples, however, those of skill in the art will appreciate the many variations of the present invention as defined by the claims appended below.

### EXAMPLES

### Example 1

### Experimental Procedures

Materials

LPCs (14:0, 16:0, 18:0, and 18:1), lysophosphatidylinositol (LPI; from liver, 80% 18:0), 18:1-LPA, 16:0-PAF, 16:0-lysoPAF, psychosine, glucosyl-β1′1-sphingosine, galactosyl-1′1-C8-ceramide, and lactosyl-β1′1-C8-ceramide were from Avanti Polar Lipids, Inc. (Alabaster, Ala.). Sphingomyelin (SM; bovine brain, mainly 18:0), C6-ceramide, sphingosine-1-phosphate (S1P) and SPC were from Toronto Research Chemicals (Toronto, ON) or Matreya, Inc. (Pleasant Gap, Pa.). D-erythro- and L-threo-SPC were from Matreya, Inc. (Pleasant Gap, Pa.). pcDNA1-C3 (encoding the C3-exoenzymne), was a kind gift from Dr. A. Wolfman, Cleveland Clinic Foundation. The PAP receptor antagonist, BN52021, was from Biomol (Plymouth Meeting, Pa.). WEB-2170 and WEB-2086 were from Boehringer Ingelheim (Ridgefield, Conn.). [³H]SPC or [3H]18:0-LPC were custom synthesized by Amersham Pharmacia Biotech, Buckinghamshire, England (68 Ci/mmol, 1 mCi/ml for [³H]SPC and 102 Ci/mmol, 1 mCi/ml for [³H]18:0-LPC). [³H]16:0-LPC (60 Ci/mmol) was purchased from American Radiolabeled Chemicals, Inc. (St Louis, Mo.).

Cell Culture

MCF10A cells (passage 34) were purchased from the Barbara Ann Karmanos Cancer Institute (Detroit, Mich.) and cultured as recommended by the provider. Experiments were performed using MCF10A cells from passage 40-46. Other cells were obtained from ATCC and were cultured either in RPMI1640 with 10% FBS or DMEM with 5% FBS (CHO and Swiss 3T3 cells).

Human RNA Master Blot Probed with GPR4

Human RNA Master Blot (Clontech, Palo Alto, Calif.) was probed with radiolabeled full-length GPR4. Briefly, the full-length GPR4 was gel purified and 25 ng was used for the synthesis of a StripAble DNA α-³²P-labeled probe (Ambion, Austin, Tex.), as per the manufacturer's instructions. The radiolabeled probe (20 ng, 20×10⁶ CPM) was hybridized to the Master Blot-in ExpressHyb hybridization solution (Clontech) overnight with continuous agitation at 65° C. The following day, the Master Blot was washed following the manufacturer's instructions and exposed to a Phospho Screen (Molecular Dynamics, Sunnyvale, Calif.).

Real-time Quantitative PCR of GPR4

Described below are examples of suitable PCR amplification schemes.

In one scheme, total RNA is extracted from tissues using the RNeasy Total RNA System (Qiagen, Valencia, Calif.). One to five micrograms of total RNA are reverse transcribed using Superscript II RT (Gibco BRL, Rockville, Md.). Eight nanograms of derived cDNA are used as a template for real-time quantitative SYBR Green I PCR. Specific primers, that have already been established and used under these same conditions, are used for each individual receptor, with GAPDH being amplified in a separate tube as a housekeeping gene. The primers for GPR4 (Genbank accession number U2105 1) are 5′-TAATGCTAGCGGCAACCACACGTGGGAG-3′ [SEQ ID NO. 1] and 5′-TCCAGTTGTCGTGGTGCAG-3′ [SEQ ID NO. 2] (230 bp product); the primers for TDAG8 are 5′-GTTGAATATTGCGATGCCGAA-3′ [SEQ ID NO. 3] and 5′-TCCTTGTTTTCCGTGGCTTTAT-3′ [SEQ ID NO. 4] (190 bp product); the primers for OGR1 are 5′-TTCGGCTACCTGCAGATCAAG-3′ [SEQ ID NO. 5] and 5′-GGTAGCGGTCCACGGAGAT-3′ [SEQ ID NO. 6] (229 bp product); and the primers for GAPDH are 5′-GAAGGTGAAGGTCGGAGT-3′ [SEQ ID NO. 7] and 5′-GAAGATGGTGATGGGATTTC-3′ [SEQ ID NO. 8] (226 bp product). The thermal cycling conditions are 95° C. for 10 minutes, followed by 40 cycles of 95° C., 15 seconds and 60 ° C., 1 minute. PCR reactions and product detection are carried out in an ABI Prism 7700 Sequence Detection System (PE Applied Biosystems, Foster City, Calif.). Amplified product is detected by measurement of SYBR Green I, which is added to the initial reaction mixture. The comparative C_{T} method is utilized to determine relative amounts of receptor. The results are expressed as a fold-change in receptor expression levels compared to a baseline sample.

In an alternative PCR scheme, total RNA was extracted from cells using the SV Total RNA Isolation System (Promega, Madison, Wis.). One to five micrograms of total RNA were reverse transcribed using Superscript II RT (Gibco BRL, Rockville, Md.). Eight nanograms of derived cDNA were used as a template for real-time quantitative SYBR Green I PCR. Primers for human GPR4 (Genbank accession number U21051) [MAKE SURE THESE ARE NOT THE SAME AS SEQ ID NOS 1-8] were 5′-TAATGCTAGCGGCAACCACACGTGGGAG [SEQ ID NO. 9] and 5′-TCCAGTTGTCGTGGTGCAG [SEQ ID NO. 10], yielding a 230 bp product. Glyceraldehyde-3-phosphate dehydrogenase (GAPDH) was amplified in a separate tube as a housekeeping gene with primers 5′-GAAGGTGAAGGTCGGAGT [SEQ ID NO. 11 ] and 5′-GAAGATGGTGATGGGATTTC [SEQ ID NO. 12], yielding a 226 bp product. Primers for mouse GPR4 were 5′-CTACCTGGCTGTGGCTCAT [SEQ ID NO. 13] and 5′-CAAAGACGCGGTATAGATTCA [SEQ ID NO. 14], yielding a 222 bp product. Mouse GAPDH was amplified with primers 5′-TGATGGGTGTGAACCAAGACA [SEQ ID NO. 15] and 5′-CCAGTGGATCAGGGATGAT [SEQ ID NO. 16]. All SYBR Green I core reagents, including AmpliTaq Gold DNA polymerase, were from PE Applied Biosystems (Foster City, Calif.). The thermal cycling conditions were 95° C. for 10 minutes, followed by 40 cycles of 95° C., 15 seconds and 60° C., 1 minute. PCR reactions and product detection were carried out in an ABI Prism 7700 Sequence Detection System (PE Applied Biosystems). The amplified product was detected by measurement of SYBR Green I, which was added to the initial reaction mixture. The threshold cycle (C_{T}) values obtained through the experiments indicate the fractional cycle numbers at which the amount of amplified target reach a fixed threshold. The C_{T} values of both target and internal reference (GAPDH) were measured from the same samples, and the expression of the target gene relative to that of GAPDH was calculated using the comparative C_{T} method. This method normalizes the expression levels and allows calculation of the relative efficiency of the target and reference amplification.

Cloning—A GPR4 PCR fragment (nucleotides #1175-1535) (Mahadevan et al. (1995) Genomics 30, 84-88) was obtained by PCR amplification using cDNA from HEY ovarian cancer cells as the template. This PCR fragment was used to screen a human genomic library (Clontech, Palo Alto, Calif.) to obtain the full-length clone of GPR4. GPR4 was subsequently cloned into mammalian expression vectors using PCR amplifications with the high fidelity Advantage cDNA polymerase (Clontech). The PCR reactions were conducted for fewer than 20 cycles and the sequence of the products was confirmed by sequencing. The primers: 5′-CAGGAATTCTCGGCAACCACACGTGGGAGG [SEQ ID NO. 17], and 5′-CGCTCTAGAGCCACTCGGGGTTCATTGTG [SEQ ID NO. 18] were used to generate full length GPR4, which was digested with EcoRI and Xba I and cloned into the pBs3HA vector (pBluescript II KS⁺ vector with three HA-tags inserted; a kind gift from Dr. J. DiDonato, Cleveland Clinic Foundation). The resulting 3HA-GPR4 was subsequently cloned into the mammalian expression vector pIRES-hygro (Clontech) to generate pIREShyg-GPR4, using primer 5′-CAGATGCATAAACGCTCAACTTTGG [SEQ ID NO. 19] and the T7 primer (inserted into the Nsi I and Not I sites of pIRES-hygro). pGPR4-GFP was generated using the T3 primer and 5′-GTCGGTACCTGTGCTGGCGGCAGCATC [SEQ ID NO. 20] (stop codon was deleted and the resulting GPR4 was cloned into Hind III and Kpn I sites of pEGFP-N1; Clontech). pSRE-Luc was purchased from Stratagene (La Jolla, Calif.). MCF10A cells were transiently transfected with pGPR4-GFP and used for calcium assays. CHO cells were transfected with pIREShyg-GPR4 (LipofectAMINE reagent; Life Technologies, Rockville, Md.) and stable clones were selected with 200 μg/ml hygromycin in DMEM/F12 plus 5% FBS. HEK293 cells were transfected with pGPR4-GFP and stable clones were selected with 400 μg/ml G418 in RPMI 1640 plus 10% FBS. Swiss 3T3 cells expressing GPR4 were derived by infection with retroviruses encoding receptor (MSCV GPR4 ires-GFP) followed by FACS sorting of GFP positive cells (Kabarowski et al. (2000) Proc Natl Acad Sci USA. 97, 12109-12114).

### Example 2

### Human RNA Master Blot Probed with GPR4 and TDAG8

GPR4 has been shown to be expressed in many human tissues. For a wider analysis of GPR4 expression in human tissues, we probed the Human RNA Master Blot (Clontech) containing RNAs from 50 different human tissues with the full length human GPR4 clone labeled with [³²P]dCTP. GPR4 showed the highest expression in ovary, liver, lung, kidney, lymph node, and sub-thalamic nucleus. Other areas of the brain had a lower expression of GPR4, as did the aorta, placenta, bone marrow, skeletal muscle, spinal cord, prostate, small intestine, and some fetal tissues. GPR4 was also expressed at a detectable level in appendix, trachea, testis, spleen, thymus, pituitary gland, adrenal gland, thyroid gland, and heart, but not in other tissues including some areas of the brain, colon, bladder, uterus, stomach, pancreas, salivary gland, mammary gland, peripheral blood leukocytes, fetal brain, and fetal heart.

To determine the tissue distribution of TDAG8, we probed the Human RNA Master Blot (Clontech) with the full length human TDAG8 clone labeled with [³²P]dCTP through PCR reactions. TDAG8 is mainly expressed in T-cell related tissues, including spleen, lymph node, thymus, and peripheral blood leukocytes. It also expresses at moderate levels in lung and small intestine tissue. In addition, we found that testis, kidney, and appendix also express moderate levels of TDAG8. TDAG8 does not express in the following tissues: whole brain, amygdale, caudate nucleus, cerebellum, cerebral cortex, frontal lobe, hippocampus, medulla oblongata, occipital pole, putaman, substantia nigra, temporal lobe, thalamus, subthalamic nucleus, spinal cord, heart, aorta, skeletal muscle, colon, bladder, uterus, prostate, stomach, ovary, pancreas, pituitary gland, adrenal gland, thyroid gland, salivary gland, mammary gland, liver, bone marrow, trachea, placenta, fetal brain, fetal heart, fetal kidney, fetal liver, fetal thymus, and fetal lung. Fetal spleen expresses TDAG8. These results suggest that TDAG8 is predominately expressed in T-cell-related tissues, with moderate expression in a limited set of other tissues.

To determine the relative expression levels of TDAG8 in different human cell lines, we have conducted real-time quantitative-PCR analysis on 19 cell lines, including five T cell lines (HBA11, HuT5, Jurkat, Jurkat-tag and Molt-4). TDAG8 expressed very high levels in T cell lines, when compared to other human cancer cells. Using the expression level of TDAG8 in HEY ovarian cancer cells as one-fold expression, five other ovarian cancer cell lines (OCC1, NIH:ovcar3, SKOV3, OVC429, and OVCA433) expressed similar levels of TDAG8 (approximately one-fold), with an exception of one ovarian cancer cell line, OVCA432, which expressed about 8-fold of TDAG8. The expression of TDAG8 in HEK293 and Hela cells was also low, but the breast cancer cell line, MCF7, expressed approximately 4.6-fold of TDAG8. Among the hematopoetic cells, HL60 and K562 expressed relatively low levels of TDAG8 (≦2-fold) and the monocyte-derived U937 expressed a relatively high level of TDAG8 (5.3-fold). In contrast to all these cell lines, T cell lines expressed very high levels of TDAG8 (25-, 67.5, 25-, 55- and 20-fold expression for HBA11, HuT5 , Jurkat, Jurkat-tag and Molt-4 cell lines, respectively). These results indicate that TDAG8 is predominately expressed in T cells.

### Example 3

### Both SPC and 16:0-LPC Induced Transient Increases in Intracellular Calcium Concentration ([Ca²⁺]ᵢ) in GPR4-transfected MCF10A Cells

Measurement of [Ca²⁺]ᵢ, was performed as described previously in Xu et al. (2000) Nat Cell Biol 2, 261-267. Briefly, pGPR4-GFP-transfected MCF10A cells were grown in specialized glass-bottom dishes (Bioptech, Inc., Butler, Pa.) and loaded with fura-2 in HEPES buffered saline. Using a dual-wavelength spectrofluorometer (RFK-6002, Photon Technology Int'l, So. Brunswick, N.J.) coupled to an inverted fluorescence microscope (Olympus, IX-70, Lake Success, N.Y.), GFP-positive cells were identified using an excitation wavelength of 488 nm, a dichroic 505 nm long-pass filter and an emitter filter at bandpass of 535 nm (Chroma Technology, Brattleboro, Vt.). Measurements of [Ca²⁺]ᵢ were performed on individual GPR4-GFP positive cells at excitation wavelengths of 340 and 380 nm and an emission wavelength of 510 nm. Conversion of the 340/380 ratio value into [Ca²⁺]ᵢ in nM was estimated by comparing the cellular fluorescence ratio with ratios acquired using fura-2 (free acid) in buffers containing known Ca²⁺ concentrations. [Ca²⁺]ᵢ was then calculated as described by Grynkiewicz et al. (1985) J Biol Chem 260, 3440-3450. All calcium assays were performed in the presence of 1 mM EGTA in the assay buffers. Therefore, intracellular calcium release, not calcium influx, was analyzed.

To test whether GPR4 is a receptor for SPC, MCF10A cells were transiently transfected with pGPR4-GFP. MCF10A cells were chosen since these cells do not respond to either SPC or 16:0-LPC in calcium assays and they express very low levels of endogenous GPR4 among many human cell lines tested.

The GFP receptor fusion was used to identify positively transfected cells, and single-cell calcium assays were performed as described in our previous studies, such as Xu et al. (2000) Nat Cell Biol 2, 261-267. SPC (1 μM) stimulated an increase in [Ca²⁺]ᵢ in GPR4-, but not vector-transfected MCF10A cells, suggesting that GPR4 is a receptor for SPC. This is further confirmed by the stereoselectivity of GPR4 favoring D-erythro-SPC (the bioactive form of SPC) vs. L-threo-SPC. Interestingly, unlike OGR1, which is specific for SPC as its ligand, GPR4-transfected cells were stimulated to produce increased [Ca²⁺]ᵢ by an additional phosphorylcholine-containing lysolipid, 16:0-LPC. To assess the affinities and potencies of SPC and 16:0-LPC, concentrations of each were varied and calcium mobilization was measured. SPC appeared to have a higher efficiency (EC₅₀=105 nM) than LPC (EC₅₀=1.1 μM), although the [Ca²⁺]ᵢ responses to LPC in GPR4-transfected cells were higher than those of SPC at greater concentrations of LPC (up to 10 μM).

LPC has been shown to activate cellular responses in a PAF receptor-dependent manner. However, LPC and SPC were not able to induce an increase in calcium through the endogenous PAF receptor in parental cells. Therefore, it is unlikely that the increase in calcium induced by LPC was mediated by a PAF receptor. Nevertheless, to confirm that LPC and/or SPC did not activate the endogenous PAF receptor in GPR4-transfected cells, three specific PAF receptor antagonists, BN52021, WEB-2170, and WEB2086, were used. Both BN52021 (200 μM) and WEB-2086 (2 μM) completely abolished the calcium signal induced by PAF (100 nM). However, the cellular calcium response to LPC or SPC was not affected, indicating that calcium increases induced by SPC and LPC were not mediated through an endogenous PAF receptor. Another PAF antagonist, WEB-2170 (2 μM), also completely blocked the action of PAF, but did not affect the increase in calcium induced by either LPC or SPC. In addition, LPC and SPC showed not only homologous, but also heterologous, desensitization to each other, suggesting that these two lipids activated the same receptor.

To determine which G protein is involved in the increased [Ca²⁺]ᵢ response to SPC and LPC in GPR4-transfected cells, the sensitivity of this activity to PTX was tested. The increase in [Ca²⁺]ᵢ response to both SPC and LPC, as well as to stimulation of endogenous LPA receptor(s), but not PAF or ATP receptors, was completely abolished by PTX (100 ng/ml, 16 h pretreatment), suggesting the involvement of a Gᵢ pathway.

In plasma, LPC is mainly present in albumin- and lipoprotein-bound forms. To determine whether BSA-bound SPC and LPC are able to induce increases in [Ca²⁺]ᵢ, we preincubated SPC (1 μM) and LPC (1 μM) with a molar excess of BSA [0.5% fatty acid-free BSA (Sigma)], for a lipid:BSA molar ratio of approximately 1:75. At this molar ratio, BSA blocked more than 50% and 95% of the increases in [Ca²⁺]ᵢ induced by SPC and LPC, respectively. These results suggest that albumin-bound LPC may not be able to activate this receptor, and support the concept of multiple LPC compartmentalization (e.g. bound and free).

Recently, Im et al have identified Psy as a ligand for TDAG8 ((2001) J Cell Biol. 153, 429-3413). TDAG8 shares approximately 38% homology with OGR1, GPR4 and G2A. To determine whether Psy is a ligand for GPR4, and to delineate the structural specificity of ligands for GPR4, we tested the effect of Psy, Glu-Sph, Gal-Cer, and Lac-Cer to increase [Ca²⁺]ᵢ in MCF10A cells. We found that at 1 μM, Psy, Glu-Sph, and Lac-Cer did not stimulate increases in [Ca²⁺]ᵢ in either MCF10A parental or GPR4-expressing cells. Gal-Cer (1 μM) induced the same level of increased [Ca²⁺]ᵢ, in both parental and GPR4-expressing MCF10A cells. These data suggest that these glycosphingolipids are unlikely to be ligands of GPR4.

### Example 4

### SPC and LPC Bind to GPR4 and TDAG8

To characterize the binding of SPC and LPC to GPR4 and TDAG8, we conducted radioligand binding assays. CHO cells were chosen for GPR4 binding assays, since HEK293 cells express relatively high levels of endogenous GPR4. CHO cells stably transfected with empty vector or GPR4 were serum starved for 20 h, then collected after exposure to 2 mM EDTA in PBS. The pelleted cells were stored at −80° C. until use. Binding assays were performed essentially as described previously (Xu et al. (2000) Nat Cell Biol 2, 261-267), except binding was performed at 4° C. Briefly, frozen cells (10⁶ cells/ml) were homogenized in a binding buffer. Assays were performed in 96-well plates in triplicate with 100 μl cell homogenate (equivalent to 10⁵ cells/well). Different amounts of [³H]SPC or [³H]16:0-LPC were added to the cell homogenates in 50 μl of binding buffer, in the presence or absence of cold SPC or 16:0-LPC, or other competitors. The plates were incubated at 4° C. for 120 min, unless otherwise indicated. Cell-bound [³H]SPC or [³H]LPC was collected onto a filter (Printed Filtermat A, Wallac, Gaithersburg, Md.) using an automated cell harvester (HARVESTER 96, Tomtec, Orange, Conn.). Specific binding was calculated by subtraction of nonspecific binding (binding detected in the presence of 100-fold excess unlabeled SPC or 16:0-LPC) from the total binding.

Cell homogenates were used for binding assays. Binding was conducted at 4° C. for 120 min or as indicated. [³H]SPC and [³H]16:0-LPC specifically bound to cell homogenates from GPR4-transfected CHO cells in a time-dependent manner and reached equilibrium after 60 min incubation at 4° C. Both CHO cells and CHO cells transfected with empty vector displayed low background binding of SPC and LPC. SPC and 16:0-LPC bindings were saturable and Scatchard analyses indicated dissociation constants (K_{d}) of 36 nM for SPC and 159 nM for LPC. The maximum binding capacities for SPC and 16:0-LPC were 996 fmole/10⁵ cells for SPC and 1,528 fmole/10⁵ cells for 16:0-LPC. SPC (p<0.001) and various LPC species (16:0, 18:0 and 18:1; p values 0.001-0.01), but not LPA (18:1), LPI(18:0), S1P, SM (18:0), 16:0-PAF or 16:0-lyso-PAF (p values >0.05), successfully competed for binding. Binding assays using [³H]18:0-LPC gave similar results. We also tested the four glycosphingolipids, Psy, Glu-Sph, Gal-Cer, and Lac-Cer, for their ability to compete for the binding of [³H]SPC and [³H]16:0-LPC to GPR4. None of these glycosphingolipids competed successfully. Thus, GPR4 was able to specifically bind both SPC and LPC (16:0, 18:0 and 18:1), with a higher affinity for SPC than LPC.

### Example 5

### Internalization of GPR4 and TDAG8 Induced by SPC, LPC, PAF, and Lyso-PAF

SPC and LPC Internalize GPR4

pGPR4-GFP stably expressing HEK293 cells were cultured in 6 cm tissue culture dishes in RPMI1640 with 10% FBS. After 16-24 h serum starvation, cells were treated with different lipids at 37° C. for 2 h. Cells were washed with cold PBS and fixed with 4% paraformaldehyde in PBS. The subcellular localization of GPR4-GFP protein was visualized under a Leica TV confocal fluorescence microscope with a 63× oil immersion lens (Wetzler, Heidelberg, Germany). The excitation and emission wavelengths were 488 nm and 515-540 nm, respectively.

G protein coupled receptors undergo agonist-dependent desensitization and internalization. When HEK293 cells are transfected with the pEGFP-N1 vector, GFP protein is expressed in the cytosol of the cells. The GPR4-GFP and TDAG8-GFP fusion proteins, on the other hand, was expressed only on the plasma membrane. One micromolar concentrations of SPC and 16:0-LPC, but not 16:0-PAF, induced internalization of GPR4 at 37° C. The PAF receptor-specific antagonist BN52021 did not block the internalization of GPR4 induced by either SPC or 16:0-LPC. Similarly, WEB-2170 and WEB-2086 did not affect the internalization of GPR4 induced by either SPC or 16:0-LPC.

SPC, PAF, and Lyso-PAF Internalize TDAG8

Using functional and binding assays, we have identified the ligands for TDAG8. Interestingly, four phospholipids containing phosphorylcholine, LPC, SPC, PAF and lyso-PAF were able to bind to TDAG8, but only SPC and LPC activated TDAG8-mediated signaling pathways [MAP kinase and serum responsive element (SRE) activation]. In addition, SPC, PAF and lyso-PAF, but not LPC induced TDAG8 receptor internalization.

### Example 6

### LPC and SPC Activated the SRE Reporter System in HEK293 Cells

The serum-response element (SRE) reporter system is a sensitive assay for receptors of lipid factors (An et al. (1998) J Biol Chem 273, 7906-7910; An et al. (1999) Mol Pharmacol 55, 787-794). The SRE reporter system used herein (pSRE-Luc) was a gift from Dr. Songzhu An (UCSF), or may be purchased from Stratagene (La Jolla, Calif.). Both systems give identical results. HEK293 and HEK293GPR4 cells were cultured in RPMI1640 with 10% FBS in 10 cm dishes to ˜85% confluence. To the cells in each dish, pSRE-Luc (10 μg) was transfected in the presence of 60 μl LipofectAMINE reagent. Cells were seeded in 96-well plates 16 h after transfection, incubated for another 24 h in RPMI1640 with 10% FBS, and starved in serum-free medium for 16 h. SPC (dissolved in PBS to 10 mM) and other lipids (LPCs were dissolved in 70% ethanol. Other lipids were dissolved in PBS, 70-95% ethanol, or 100% MeOH) were diluted in serum free RPMI 1640 and added to the cells, followed by a 10 h incubation. Luciferase activity was measured in Microlite™ 1 plates (DYNEX Technologies, Inc., Virginia) using 60 μl of cell lysate and 20 μl luciferase substrate. PTX (100 ng/ml) was added during the 16 h serum starvation period and pcDNA1-C3 (encoding the C3-exoenzyme, 2 μg) was co-transfected with pSRE-Luc (10 μg). All experiments were performed in quadruplicate and were repeated at least three times. The Student's t test was performed using the GraphPad Instat software (San Diego, Calif.). p<0.05 was considered to be statistically significant.

Using the luciferase assay, vector-transfected HEK293 cells transfected with the SRE reporter system responded to SPC (1 μM), but not 16:0-LPC, with ≦1.5-fold activation. Activation was increased 3.1- and 4-fold, respectively, in response to 16:0-LPC (1 μM) and SPC (1 μM) in both GPR4- and TDAG8-transfected HEK293 cells that were also transfected with the SRE reported system. These increases were statistically significant (p<0.001) when compared to the responses in vector-transfected cells. In contrast, although LPA and S1P induced significant transcriptional activation of SRE in vector-transfected HEK293 cells, this activation was not altered by GPR4 or TDAG8 cotransfection. In addition, we tested other phosphorylcholine-containing lipids, including 16:0-PAF, 16:0-lyso-PAF and 18:0-SM, and found that none of them induced significant transcriptional activation of SRE.

The SRE transcriptional activity in response to SPC, but not LPC, in parental HEK293 cells, can be explained by the endogenous expression of GPR4 in HEK 293 cells and the relatively lower affinity of GPR4 for LPC compared to SPC. GPR4 transfection enhanced the activation of SRE reporter by both SPC and LPC. EC₅₀ values for the activation of SRE were 63 nM for SPC and 160 nM for 16:0-LPC. The differences in EC₅₀ values obtained using SRE activation from those using the calcium assay (105 nM and 1.1 μM for SPC and LPC, respectively) are possibly derived from different coupling efficiencies of distinct signaling pathways and/or different cellular environments.

To determine which G protein and other signaling intermediates might be involved in the activation of SRE by SPC and 16:0-LPC, we pretreated cells with PTX (100 ng/ml) for 16 h, or co-transfected the specific Rho inhibitor C3exoenzyme (1.5 μg pcDNA3-C3), with the reporter system. Both PTX and C3-exoenzyme partially inhibited SRE-reporter activation. When the two inhibitors were added together, SRE-reporter activation in response to either SPC or 16:0-LPC was almost completely blocked, suggesting that Gᵢ and Rho signaling pathways were involved in SRE activation through GPR4.

### Example 7

### SPC and LPC Activated ERK MAP Kinase in a GPR4-dependent Manner

MAP kinases are key signaling intermediates of DNA synthesis and cell proliferation. Swiss 3T3 cells were infected with MSCV GPR4-ires-GFP or MSCV ires-GFP, and subsequently cells sorted by FACS for positive expression of GFP as described previously (Kabarowski et al. (2000) Proc Natl Acad Sci USA. 97, 12109-12114). Cells were plated in 6-well plates in DMEM containing 5% FBS, serum-starved overnight, and then treated lipids in DMEM for the indicated times. Cells were lysed on ice in RIPA buffer containing 50 mM Tris-HCl, pH 7.5, 1% Nonidet P-40, 0.5% deoxycholate,0.1% SDS, 150 mM NaCl, 2 mM EGTA, 25 mM sodium fluoride, 1 mM sodium orthovanadate, and 1× protease inhibitors (Sigma P8340). Lysates containing equal amounts of protein were separated on 10% SDS-polyacrylamide gels and transferred onto nitrocellulose membranes. Antibodies against phosphorylated ERK1/2 (Cell Signaling Technologies; Beverly, Mass.) were used to probe the membrane and the ECL system (Amersham) was used for detection. To normalize the amounts of protein loaded in each lane, membranes were stripped and re-probed with antibodies against total ERK (Cell Signaling Technologies). In some experiments, cells were pretreated with 100 ng/ml PTX for 12-16 h prior to SPC and LPC stimulation.

To determine whether GPR4 mediates ERK MAP kinase activation in response to SPC and LPC, we conducted Western blot analyses of GPR4-ires-GFP-Swiss 3T3 and GFP-Swiss 3T3 cells treated with SPC, 16:0-LPC, and a number of other lipids. The parental and GFP infected Swiss 3T3 cells showed a basal level of ERK activation, as detected by anti-phospho-ERK antibody. SPC (100 nM) increased this level of activation. In GPR4-ires-GFP-infected Swiss 3T3 cells, both SPC (100 nM) and LPC (100 nM) enhanced ERK activation, and SPC was more potent than LPC. A number of other lipids, including S1P, Lac-Cer and PAF, also activated ERK in Swiss 3T3 cells, but activation was independent of GPR4 expression. Lipid stock solutions, dissolved in ethanol or MeOH, were ≧10 mM. Since the highest final concentration of lipids used in this study was 10 μM, the solvent content was ≦0.1% in any experiment. We routinely performed solvent controls and found that at final solvent concentrations of <??0.1%, 70-100% ethanol and 100% methanol did not alter any parameters tested.

The higher potency of SPC over LPC was further reflected in the concentration- and time-dependent ERK activation. ERK activation induced by SPC compared to that by LPC was evident at a lower concentration (approximately 10 nM vs. 100 nM), at earlier time points (1 min vs. 5 min), and was maintained for a longer time. These results strengthen the notion that both SPC and LPC are ligands for GPR4, but SPC has a higher affinity than LPC for GPR4.

In GPR4-infected Swiss 3T3 cells, SPC-induced ERK activation was sensitive to PTX, suggesting involvement of Gᵢ signaling. This is in contrast to ourprevious studies where SPC induced ERK activation via a PTX-insensitive pathway in OGR1-transfected HEK293 cells (Xu et al. (2000) Nat Cell Biol 2, 261-267). To determine whether this difference was due to receptor subtype or different cell lines used, we tested the PTX-sensitivity of SPC-induced ERK activation in OGR1-infected Swiss 3T3 cells. Our results demonstrated that in Swiss 3T3 cells, SPC-induced ERK activation via OGR1 was PTX-insensitive. Thus, although GPR4 and OGR1 are highly homologous, the same high-affinity ligand (SPC) induces activation of ERK through a different G protein pathway for each receptor.

To further explore the TDAG8-mediated cellular response to SPC, MAP kinase activation was examined using the MAP kinase reporter system from Stratagene. In HEK293 cells co-transfected with TDAG8 and the reporter system, LPC and SPC (1-5 μM) induced activation of p42/44 MAP kinase as revealed by the reporter luciferase activity. SPC and LPC showed similar potency to active MAP kinase in TDAG8-transfected cells. In OGR1 co-transfected cells, the MAP kinase activation was only achieved by SPC. LPC, on the other hand was weakly inhibitory.

### Example 8

### SPC Stimulated DNA Synthesis in GPR4-infected Swiss 3T3 Cells

The effect of SPC and LPC on DNA synthesis was measured using [³H] thymidine incorporation. Briefly, GPR4-ires-GFP- and GFP-Swiss 3T3 cells were plated in 96-well plates, serum-starved for 24 h, and treated with SPC, LPC, or other lipids in serum-free DMEM for 24 h. Cells were incubated with 0.75 μCi/ml [³H]thymidine in serum-free DMEM for the last 18 h. Cells were harvested onto filter papers presoaked in 1% polyethylenimine using the automated cell harvester HARVEST 96. Incorporated [³H]thymidine was counted in a 1450 Microbeta Trilux Liquid Scintillation and Luminescence Counter (Perkin-Elmer-Wallac, Inc.).

To determine whether SPC and LPC affect DNA synthesis in a GPR4-dependent fashion, we measured [³H]thymidine incorporation into GPR4-ires-GFP-Swiss 3T3 and GFP-Swiss 3T3 cells. SPC stimulated DNA synthesis in both parental and GFP-infected cells (approximately 6.3-fold increase with 3 μM SPC). This stimulation was further enhanced by the expression of GPR4 (1.8- to 2-fold increase over GFP-infected Swiss 3T3 cells). In both GFP- and GPR4-GFP expressing cells, DNA synthesis stimulated by SPC was inhibited by PTX (100 ng/ml for 16 h prior to lipid treatment), suggesting Gᵢ signaling was required for this activity. GFP-expressing cells did not respond significantly to 16:0-LPC, whereas [³H]thymidine incorporation increased 1.6-fold in GPR4-infected Swiss 3T3 cells in response to 3 μM 16:0-LPC. Higher concentration of lipids did not further increase [³H]thymidine incorporation stimulated by SPC or LPC.

### Example 9

### SPC and LPC Induce Cell Migration in a GPR4- and TDAG8-dependent Manner

Chemotaxis was measured in a modified Boyden chamber assay. Briefly, different lipids were added to the lower chambers. GPR4-ires-GFP- and GFP-Swiss 3T3 cells were serum starved for 4 h, trypsinized, and seeded in the upper chambers of Boyden-transwell plates (Corning Inc., Corning, N.Y.). The chambers were incubated for 6 to 8 h. The number of cells that migrated to the lower face of the membrane was counted in 4 random fields. Data are represented as the average ±SD of three independent experiments. For the chemokinetic assay, the same concentrations of lipids were added to both the upper and lower chambers. For Rho inhibition studies, C3-exoenxyme was transiently transfected into Swiss 3T3 cells and cell migration assays were performed 48 hours later.

As a major component of oxidized low-density lipoprotein (ox-LDL), LPC has been proposed to play a role in atherosclerotic lesion development (Lusis, A. J. (2000) Nature 407, 233-341; Chisolm et al. (2000) Free Radic Biol Med 28, 1697-1707). One of the roles of LPC potentially related to atherosclerosis is as a chemoattractant for monocytes, T lymphocytes, and smooth muscle cells (Prokazova et al. (1998) Biochemistry (Moscow) 63, 31-37; Kohno et al. (1998) Circulation 98, 353-359; McMurray et al. (1993), J Clin Invest. 92, 1004-1008). We used Swiss 3T3 cells infected with GFP or GPR4-ires-GFP as a model system to compare the effects of SPC and 16:0-LPC on cell migration. GPR4 overexpression in Swiss 3T3 fibroblasts increased cell migration in response to SPC (100 nM; lower chamber only) and 16:0-LPC (100 nM; lower chamber only) 2.0-fold and 1.7-fold, respectively, over that observed in GFP-Swiss 3T3 cells. Other lipids (18:1-LPA, S1P, or 16:0PAF) did not alter cell migration in GPR4- vs. vector-transfected cells. Cell migration stimulated by both SPC and LPC was inhibited by C3-exoenzyme expression, suggesting that Rho is involved in this process.

Concentration response studies indicate that SPC and LPC were effective at inducing cell migration in the 1-100 nM concentration range. To determine whether this effect was chemotactic or chemokinetic, we measured cells that migrated from the upper to the lower chambers in Boyden chamber assays, conducted with lipids (at 100 nM) in both upper and lower chambers. SPC or 16:0-LPC did not significantly change cell motility when compared to controls (without lipid in either chamber) in either GFP or GFP-GPR4 expressing Swiss 3T3 cells. S1P slightly inhibited, PAF slightly enhanced, and LPA did not show a significant effect on cell migration in treated vs. untreated GFP or GFP-GPR4 expressing cells. These results suggest that the effect of SPC and 16:0-LPC on cell migration was chemotactic, not chemokinetic, and that the chemotactic effect was mediated through GPR4.

LPC functions as a chemoattractant for macrophage and T cells. The attachment of monocytes and T-lymphocytes to the injured endothelium followed by their migration into the intima is one of the first and most crucial steps in lesion development, which may trigger cell-mediated immunity to atherogenesis.

To determine whether GPR4 and/or TDGA8 can mediate the LPC-induced cell migration, we transfected HEK293 cells with GPR4 or TDAG8. Cell migration was performed in Transwell apparatus. GPR4-transfected HEK293 cells, and TDAG8-transfected HEK293 cells migrated to the low-phase of the membrane and the lower chamber of the Transwell in response to SPC and LPC, used at 0.2 μM. This cell migration experiment demonstrated increased cell migration in response to LPC or SPC in cells in a GPR4- and TDAG8-dependent manner.

Our experiments indicate that LPC and SPC may act as chemoattractants through TDAG8. When TDAG8 was overexpressed in HEK293 cells, cell migration in response to LPC and SPC was increased four to five-fold, when compared with control cells.

### Example 10

### LPC Upregulates GPR4 and TDAG8 in Jurkat Cells

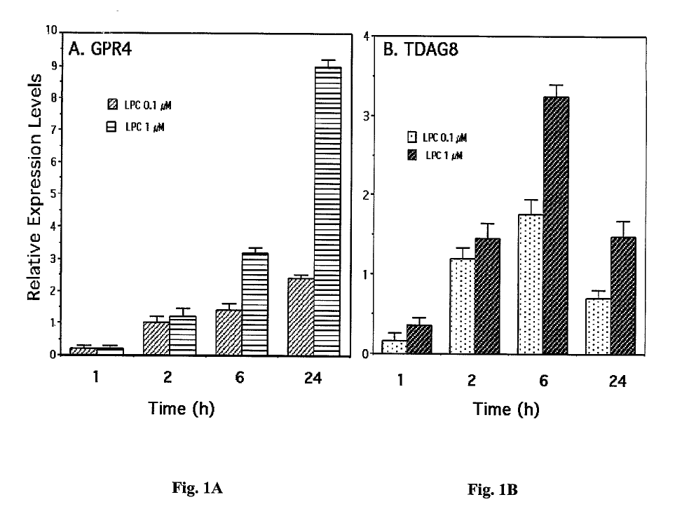

Jurkat cells were treated with 0.1 and 1 μM LPC for up to 24 hours followed by expression analysis of GPR4 and TDAG8 levels with real-time quantitative RT-PCR. A time- and dose-dependent up regulation of both GPR4 and TDAG8 was observed, as shown in FIG. 1. This indicates that increased levels of LPC are able to upregulate its receptors in order to enhance its cellular effects, which could possibly contribute to the metastatic and angiogenic potentials of the cells.

### Example 11

### SPC Levels were Elevated in Ascites from Patients with Ovarian Cancer

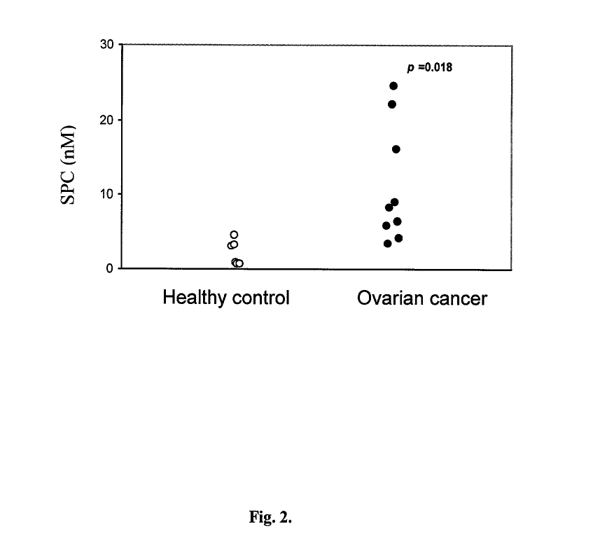

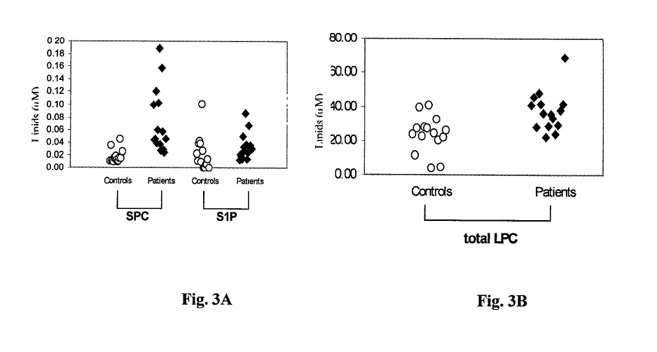

We have recently developed an ESI-MS based method to detect all lyso-phospholipids in body fluids (Xiao et al., Ann N Y Acad Sci. 905: 242-59., 2000; Xiao et al., Anal Biochem. 290: 302-313, 2001). Using this method, we have analyzed lipid contents in plasma, ascites and peritoneal washings. The results shown in FIG. 2 and FIG. 3 demonstrate that SPC was elevated in plasma and ascites from patients with ovarian cancer, when compared with healthy controls or patients with benign diseases.

### Example 12

### Construction of a Synthetic Peptide that Binds to SPC

As shown above, SPC is elevated in plasma and ascites from patients with ovarian cancer. The concentrations of SPC in plasma and ascites are not high enough to have growth inhibitory effect. However, SPC under these concentrations may play other pathological roles. We have shown that SPC at the concentrations present in ovarian cancer ascites may synergize with pathological concentrations of LPA to stimulate interleukin-8 (IL-8) secretion. IL-8 is a pro-inflammatory and pro-angiogenic factor. It is present at relatively high concentrations in ascites from patients with ovarian cancer. It has been shown that the expression level of IL-8 directly correlates with the progression of human ovarian carcinomas implanted into the peritoneal cavity of nude mice, and the survival of mice bearing tumors derived from human ovarian cancer cells is inversely associated with the expression of IL-8 (Yoneda et al., Journal of the National Cancer Institute. 90: 447-54, 1998). In addition, abnormal elevation of SPC has been shown to be associated with certain pathological conditions, such as Niemann-Pick disease type A or atopic dermatitis. Therefore, eliminating or reducing SPC abnormal concentrations is expected to be beneficial to these diseases. We have identified a peptide from a phage library that binds to SPC specifically.

Biotinylation of SPC

One milligram of SPC and 3 mg of EZ-Link Sulfo-NHS-LC-Biotin (Pierce) were mixed in 0.1 M phosphate buffer (pH 7.0). After 30 minutes of mixing/rocking at room temperature, the mixture was extracted twice with butanol. The butanol phase was combined and dried under nitrogen. The dried residue was reconstituted in chloroform and the insoluble part was filtrated out. The chloroform phase was dried and the residue was dissolved in methanol. The identity and purity of the product was determined by ESI-MS and MS/MS. The quantity of the product (biotinylated SPC) was obtained by MRM-MS mode using SPC as an internal standard.

Biopanning

Biotinylated SPC (10 nM) was mixed with 10 1 Ph.D.-C7C phage library (from New England BioLabs) in TBST (0.1% Tween 20). The mixture was gently mixed for one hour at room temperature. The mixture was transferred to a streptavidin coated dish (Pierce), which was blocked by BSA. After 10 minute incubation at room temperature, 0.1 mM biotin was added to the dish and the dish was incubated for another 5 minutes. After the dish was washed 10 times with TBST, the bound phage were eluted by adding 1 mM SPC (1 h incubation at room temperature). The eluted phage were amplified and used to repeat the above procedure for the second to the fourth round of biopanning. After the fourth biopanning, the eluted phage were filtered and 16 single clones were picked up for amplification.

Phage Clone #414 Blocked SPC-stimulated Growth Inhibition in HEY Cells

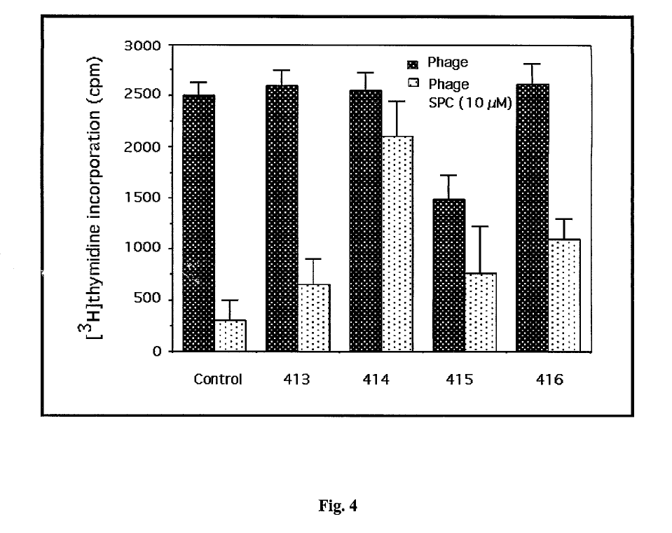

To determine if these selected phage clones were capable of blocking a biological effect of SPC, we mixed phage preparations with SPC. After a 30 min incubation, this mixture was added to HEY cells and its effect on SPC-induced cell proliferation was tested. If a phage clone was carrying a specific sequence which binds to SPC, the addition of the phage to SPC could block the effect of SPC on HEY cells. FIG. 4 shows that one of the phage clones, #414, was able to inhibit the growth inhibitory effect of SPC on HEY cells, suggesting that the peptide carried by clone 414 is able to bind to SPC specifically.

The sequence from Clone 414 is: Cys-His-Thr-Gln-Ala-Asp-Ser-Cys [SEQ ED NO. 21 ] with a disulfide bridge between the two cys residues.

All patents, patent applications, and documents cited herein are incorporated by reference as if fully set forth herein. As those skilled in the art will appreciate, numerous changes and modifications may be made to the invention without departing from the spirit thereof. It is intended that all such variations fall within the scope of the invention.

We claim:

1. A method of suppressing tumor cell growth comprising contacting the tumor cell with an antagonist of GPR4 or TDAG8.

2. The method of claim 1 wherein said antagonist is a synthetic peptide which binds to SPC.

3. The method of claim 1 wherein said synthetic peptide is the peptide of SEQ ID NO. 21.

4. The method of claim 1 performed in vivo in a human.

5. A method of treating a disease condition in a patient comprising administering to the patient a therapeutically effective amount of an antagonist GPR4 or TDAG8.

6. The method of claim 5 wherein said antagonist is a synthetic peptide which binds to SPC.

7. The method of claim 6 wherein said synthetic peptide is the peptide of SEQ ID NO.21.

8. The method of claim 6 wherein the disease is selected from the group consisting of Niemann-Pick disease type A and atopic dermatitis.

9. A method of treating a disease condition in a patient comprising administering to the patient a therapeutically effective amount of an agent which interferes with GPR4 or TDAG8 binding to LPC.

10. The method of claim 9 wherein the disease condition is atherosclerosis, arthritis, liver cirrhosis, endometriosis, cancer, and Alzheimer's disease.

11. The method of claim 10 wherein the agent is lyso-PAF.

12. A method of preventing a disease condition comprising administering to the patient a therapeutically effective amount of an agent which interferes with GPR4 or TDAG8 binding to LPC.

13. The method of claim 12 wherein the disease condition is an inflammatory disease condition selected from the group consisting of atherosclerosis, arthritis, liver cirrhosis, endometriosis, cancer, or Alzheimer's disease.

14. The method of claim 13 wherein the agent is lyso-PAF.

15. A method of detecting the presence of a disease condition in a patient comprising measuring the level of SPC in the patient.

16. The method of claim 15 wherein the disease is ovarian cancer.

17. A method of determining the progress of a disease condition in a patient comprising measuring the level of SPC in the patient.

18. The method of claim 17 wherein the disease is ovarian cancer.

19. A method of determining whether a disease condition in a patient is benign comprising measuring the level of SPC in the patient.

20. The method of claim 19 wherein the disease is ovarian cancer.

21. A method of modulating the activity of GPR4 comprising contacting the GPR4 with SPC or LPC.

22. A method of modulating the activity of TDAG8 comprising contacting the TDAG8 with SPC or LPC.

23. A method of screening a drug candidate comprising contacting the drug candidate with GPR4 or TDAG8 in the presence of SPC or LPC.

24. A composition comprising a synthetic peptide capable of binding to SPC.

25. The composition of claim 24 which is capable of interfering with the binding of SPC to a GPCR.

26. The composition of claim 25 wherein the GPCR is selected from the group consisting of OGR1, G2A, GPR4, and TDAG8.

27. The composition of claim 24, further comprising a pharmaceutically acceptable excipient.
